SEQUENCE LISTING (3 sequences)

SEQ ID NO: 1 - DNA, 300 bp, Homo sapiens
gaggcagcttttcctgcttacacaatacagaaatatgatttcaaaaatctattaaaattttattaatctcagaaggcatgatttctaattgtgtttgatcttacacttgttatgatttaggaattcacatctgagttggttgcatgatgctatagttggcaacatgaytctgaccgccaccatcacaaatagaggttagaaaatattacttatgtgaaaataaatgccatttctggcacctaaaacagctcttttctcaccttcctatgatgaggttttattgagcttttgcaggaaaga

SEQ ID NO: 2 - DNA, 20 bp, Artificial sequence
  Synthetic primer
ggcagcttttcctgcttaca

SEQ ID NO: 3 - DNA, 20 bp, Artificial sequence
  Synthetic primer
tctttcctgcaaaagctcaa

(19) United States
(12) Patent Application Publication
(10) Pub. No.: US 2011/0028453 A1
(43) Pub. Date: Feb. 3, 2011

(54) Treating Negative Symptoms of Schizophrenia Associated with Defective Neuregulin 1

(76) Inventors: Wen-Mei Fu, Taipei City (TW); Ya-Hsuan Chiang, Taichung County (TW); Hai-Gwo Hwu, Taipei City (TW); Chih-Min Liu, Taipei City (TW)

Correspondence Address: OCCHIUTI ROHLICEK & TSAO, LLP, 10 FAWCETT STREET, CAMBRIDGE, MA, 02138, US

(21) Appl. No.: 12/843,961

(22) Filed: Jul. 27, 2010

Related U.S. Application Data
(60) Provisional application No. 61/230,140, filed on Jul. 31, 2009.

(51) Int. Cl. A61K 31/55; A61K 31/137; A61K 31/15; A61K 31/381; A61K 31/343; A61P 25/18; C12Q 1/68

(52) U.S. Cl. 514/217; 514/654; 514/640; 514/438; 514/469; 435/6

(57) ABSTRACT

Use of a compound that is a serotonin transporter inhibitor, a selective norepinephrine reuptake inhibitor, or a 5-HT_{1A} agonist for alleviating negative symptoms in a schizophrenia patient who carries a defective neuregulin 1 gene.

### CROSS-REFERENCE TO RELATED APPLICATION

This application claims the benefit of U.S. Provisional Application No. 61/230,140, filed on Jul. 31, 2009, the contents of which are hereby incorporated by reference in its entirety.

### BACKGROUND OF THE INVENTION

Schizophrenia is a complex mental disorder affecting 0.5-1% of the general population worldwide. Patients suffering from schizophrenia typically experience positive symptoms (e.g., hallucinations, delusions, and racing thoughts), negative symptoms (e.g., apathy, lack of emotion, poor or nonexistant social functioning), or cognitive symptoms (e.g., disorganized thoughts, difficulty inconcentrating or following instructions, and memory problems).

Both genetic and environmental factors contribute to the development of schizophrenia. To date, several candidate genes have been identified as associated with schizophrenia, including DTNBP1, NRG1, G72/G30, and TRAR4. Social stress, family stress, and other environmental factors have also been suggested for triggering this mental disorder.

### SUMMARY OF THE INVENTION

The present invention is based on unexpected discoveries that NRG1^{+/-} mice are more sensitive to serotonin transporter inhibitors (i.e., desipramine, imipramine, venlafaxine duloxetine, fluvoxamine and escitalopram) in behavior changes as compared to their wild-type counterparts.

Accordingly, one aspect of this invention features a method for alleviating a negative symptom in a schizophrenia patient carrying a defective NRG1 gene by administering to such a patient an effective amount of a compound (10 to 600 mg/day) that is a serotonin transporter inhibitor (i.e., a selective serotonin transporter inhibitor or a serotonin-norepinephrine transporter inhibitor), a selective norepinephrine reuptake inhibitor, or a 5-HT_{1A} receptor agonist.

A schizophrenia patient who are defective in NRG1 can be identified by examining the sequence of his or her NRG1 gene (e.g., the sequence of the NRG1 promoter), a single nucleotide polymorphism (SNP) in the NRG1 gene (e.g., at position 168 in SEQ ID NO:1), or the NRG1 protein or mRNA levels.

Examples of serotonin transporter inhibitors to be used in the method of this invention include, but are not limited to, citalopram, dapoxetine, escitalopram, fluoxetine, fluvoxamine, indalpine, paroxetine, sertraline, zimelidine, desvenlafaxine, duloxetine, levomilnacipran, milnacipran, venlafaxine, amitriptyline, butriptyline, clomipramine, desipramine, dosulepin, doxepin, imipramine, lofepramine, nomifensine, nortriptyline, protriptyline, sibutramine and trimipramine. Examples of selective norepinephrine reuptake inhibitors include amineptine, atomoxetine, bupropion, dexmethylphenidate, mazindol, methylphenidate, reboxetine, nisoxetine, and viloxazine. Examples of 5-HT_{1A} receptor agonists (e.g., partial agonists), include buspirone, flesinoxan, gepirone, and ipsapirone. All of the inhibitors/agonists disclosed herein refer to either the corresponding compounds or their pharmaceutically acceptable salts.

Another aspect of the present invention features a method for identifying a schizophrenia patient suitable for the treatment described above. This method includes at least the following steps: (i) examining the neuregulin 1 gene function in a schizophrenia patient displaying a negative symptom, and (ii) assessing whether the patient is suitable for the treatment of this invention based on the neuregulin 1 gene function in the patient. Presence of a defective neuregulin 1 gene indicates that the patient is suitable for the treatment. In one example, the examining step is performed by determining the protein or mRNA level of neuregulin 1 in the patient. A lower protein or mRNA level relative to that in a person carrying a wild-type neuregulin 1 gene indicates that the patient carries a defective neuregulin 1 gene. Alternatively, the examining step is performed by determining the neuregulin 1 activity in the patient, a lower neuregulin 1 activity relative to that of a wild-type neuregulin 1 gene indicating that the patient carries a defective neuregulin 1 gene. In yet another example, the examining step is performed by determining the sequence of the neuregulin 1 gene in the patient. Presence of a mutation that affects neuregulin 1 activity indicates that the patient carries a defective neuregulin 1 gene. The examining step can also be performed by determining the sequence of a promoter region in the neuregulin 1 gene in the patient or the single nucleotide polymorphism (SNP) at position 168 in SEQ ID NO:1. Presence of a mutation in the promoter region that affects promoter activity or presence of TT at SNP168 indicates that the patient carries a defective neuregulin 1 gene.

Also within the scope of this invention are (i) a pharmaceutical composition for alleviating a negative symptom of a schizophrenia patient, the composition containing a serotonin/norepinephrine transporter inhibitor, and (ii) use of the inhibitor in manufacturing a medicament for alleviating the negative symptom.

The details of one or more embodiments of the invention are set forth in the description below. Other features or advantages of the present invention will be apparent from the following drawings, detailed description of two examples, and also from the appended claims.

### BRIEF DESCRIPTION OF THE DRAWINGS

The drawings are first described.

FIG. 1 is a chart showing the effects of serotonin transporter inhibitors venlafaxine, imipramine and desipramine on immobility of both wild-type and NRG1^{+/-} mice. *: P<0.05; **: P<0.01.

FIG. 2 is a diagram showing the effects of serotonin transporter inhibitors venlafaxine, imipramine, desipramine, duloxetine, fluvoxamine and escitalopram on social behavior of both wild-type and NRG1^{+/-} mice. Panel A: male mice treated with venlafaxine, imipramine, desipramine, duloxetine, fluvoxamine and escitalopram. Panel B: female mice treated with duloxetine and escitalopram. #: P<0.05; ** or ##: P<0.01.

### DETAILED DESCRIPTION OF THE INVENTION

Described herein is a method for alleviating a negative symptom in a schizophrenia patient carrying a defective NRG1 gene with an effective amount of a serotonin transporter inhibitor, a selective nonepinephrine reuptake inhibitor, or a 5-HT_{1A} agonist. A defective NRG1 gene is a mutated neuregulin 1 gene that either expresses a lower level of neuregulin 1 protein as compared to a wild-type neuregulin 1 gene (e.g., the neuregulin 1 gene described under GenBank accession number CN603655, CN603656, CN603657, CN603658, CN603652, CN603653, CN603654, or NM_{—}013962.2), or encodes a mutated neuregulin 1 protein with reduced activity as compared to a wild-type neuregulin 1 protein (e.g., the neuregulin 1 protein described under GenBank accession number ABR13844.1, ABR13843.1, ABR13842.1, ABQ53543.1, ABQ53541.1, ABQ53542.1, ABQ53540.1, ABQ53539.1, NP_{—}039256.2, or AAM71140.1).

A schizophrenia patient who is defective in NRG1 can be identified via a conventional method. In one example, the NRG1 gene or a fragment thereof (e.g., its promoter region) can be amplified from a candidate patient by PCR and subjected to sequencing analysis. Upon comparing the gene/promoter sequence thus obtained with that of a wild-type NRG1 gene, whether the candidate patient carries a defective NRG1 gene can be determined. In another example, a particular SNP (e.g., the SNP at position 168 in SEQ ID NO:1 shown below; see also Example 3 below) within the NRG1 gene can be used as a marker indicating presence of a functional/defective NRG1 gene (typeV).

(SEQ ID NO: 1)
GAGGCAGCTT TTCCTGCTTA CACAATACAG AAATATGATT
TCAAAAATCT ATTAAAATTT TATTAATCTC AGAAGGCATG
ATTTCTAATT GTGTTTGATC TTACACTTGT TATGATTTAG
GAATTCACAT CTGAGTTGGT TGCATGATGC TATAGTTGGC
AACATGA [symbol] TC TGACCGCCAC CATCACAAAT AGAGGTTAGA
AAATATTACT TATGTGAAAA TAAATGCCAT TTCTGGCACC
TAAAACAGCT CTTTTCTCAC CTTCCTATGA TGAGGTTTTA
TTGAGCTTTT GCAGGAAAGA
Y: T or C

Alternatively, the mRNA or protein level of NRG1 can be determined in a candidate patient to assess whether the patient is NRG1 defective.

After a schizophrenia patient has been determined for carrying a defective NRG1 gene, an effective amount of a serotonin transporter inhibitor, a selective norepinephrine reuptake inhibitor, or a 5-HT_{1A} agonist can be administered to the patient to reduce his or her negative symptom. As used herein, an effective amount refers to the amount of each active agent required to confer therapeutic effect on the subject, either alone or in combination with one or more other active agents. Effective amounts vary, as recognized by those skilled in the art, depending on route of administration, excipient usage, and co-usage with other active agents.

Serotonin transporter inhibitors (SRIs) are a well-known family of drugs that block the activity of serotonin or norepinephrine transporter, thereby suppressing reuptake of serotonin or norepinephrine. This family of drugs include selective serotonin reuptake inhibitors (e.g., citalopram, dapoxetine, escitalopram, fluoxetine, fluvoxamine, indalpine, paroxetine, sertraline, and zimelidine) and serotonin-norepinephrine reuptake inhibitors (e.g., amitriptyline, venlafaxine, desvenlafaxine, imipramine, desipramine, duloxetine, milnacipran, levomilnacipran, sibutramine, butriptyline, clomipramine, dosulepin, doxepin, lofepramine, nortriptyline, protriptyline and trimipramine).

Selective norepinephrine reuptake inhibitors (e.g., amineptine, atomoxetine, bupropion, dexmethylphenidate, mazindol, methylphenidate, reboxetine, nisoxetine and viloxazine) specifically block norepinephrine transporter, thereby selectively suppressing reuptake of norepinephrine.

5-HT_{1A} agonists, such as azapirones (e.g., buspirone, flesinoxan, gepirone, and ipsapirone), are compounds that mimic the effect of serotonin and activates serotonin receptor 5-HT_{1A}.

To practice the method of the present invention, any of the above-described serotonin transporter inhibitors, selective norepinephrine reuptake inhibitors, or 5-HT_{1A} agonists can be mixed with a pharmaceutically acceptable carrier to form a pharmaceutical composition. The carrier in the pharmaceutical composition must be “acceptable” in the sense of being compatible with the active ingredient of the formulation (and preferably, capable of stabilizing it) and not deleterious to the subject to be treated. For example, solubilizing agents such as cyclodextrins, which form specific, more soluble complexes with the inhibitor, or one or more solubilizing agents, can be utilized as pharmaceutical excipients for delivery of the inhibitor. Examples of other carriers include colloidal silicon dioxide, magnesium stearate, cellulose, sodium lauryl sulfate, and D&C Yellow # 10.

The pharmaceutical composition mentioned above can be administered to a schizophrenia patient via a conventional route, e.g., orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

The pharmaceutical composition described herein can also be administered in the form of suppositories for rectal administration.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference.

### Example 1

### Correlation Between NRG1 and Serotonin or Norepinephrine Transporter

### (i) NRG1β Reduced Expression of Both Serotonin and Norepinephrine Transporters in Astrocytes and Neurons

A172 cells (originating from a human brain glioma), SH-SY5Y cells (a human neuroblastoma cell line), primary astrocytes, and primary neurons were used in this study.

A172 cells were cultured in DMEM supplemented with 10% heat-inactivated fetal bovine serum (FBS; Hyclone, Logan, Utah), 100 U/mL penicillin and 0.1 mg/mL streptomycin (Invitrogen, Carlsbad, Calif.) at 37° C. in a humidified incubator with 5% CO₂ and 95% air.

SH-SY5Y cells were maintained in F12/MEM supplemented with 10% FBS, 100 U/mL penicillin and 0.1 mg/mL streptomycin (Invitrogen, Carlsbad, Calif.) at 37° C. in a humidified incubator with 5% CO₂ and 95% air.

Rat primary astrocytes were prepared as follows. Cortical tissues were obtained from E17 Sprague-Dawley rats. Cells in the tissue were isolated and suspended in DMEM supplemented with 10% FBS, 100 U/mL penicillin and 0.1 mg/mL streptomycin. The cells (1×10⁷) were then seeded in a 75-cm² flask and cultured in a humidified chamber at 37° C. with 5% CO₂ for 7 days with medium changed every 3 days. On day 7, the flask was placed on a shaker platform and shaken at 220 rpm for 6 hrs at 37° C. to remove the oligodendrocytes/microglia in the cultures, thereby enriching astrocytes. The enriched astrocytes were then seeded in a 6-well plate.

Rat primary neurons were prepared from cortex of E17 Sprague-Dawley rats as follows. Briefly, cortex tissues were obtained from the rats, treated to remove meningeal tissue, minced and mechanically dissociated by passage through a flame-polished Pasteur pipette. The cells thus obtained were suspended in DMEM supplemented with 10% FBS, 100 U/mL penicillin, and 0.1 mg/mL streptomycin and were seeded at 1×10⁶ cells/well on poly-D-lysine-coated 6-well plate. 24 hours after seeding, the culture medium was replaced with DMEM supplemented with 2% B27 (Invitrogen, Carlsbad, Calif.), 100 U/mL penicillin, and 0.1 mg/mL streptomycin. The enriched cortical neurons thus prepared were maintained in a humidified chamber at 37° C. in a 5% CO₂ atmosphere for 7 days, the medium being changed every 3 days.

The A172 cells, SH-SY5Y cells, primary astrocytes, and primary neurons mentioned above were treated with NRG1β at various concentrations (i.e., 1, 3, 10, or 30 ng/ml) for 24 hours. Cellular proteins were isolated from these cells and analyzing by the Western blotting assay described below to detect their levels of serotonin transporter.

The cells were lysed and the lysates were suspended in a RIPA buffer (50 mM HEPES (pH 7.4), 4 mM EDTA, 150 mM NaCl, 10 mM Na₄P₂O₇, 100 mM NaF, 2 mM Na₃VO₄, 1% Triton X-100, 0.25% sodium deoxycholate, 50 mM 4-(2-aminoethyl) benzene sulfonylfluoride, 50 μg/mL leupeptin, and 20 μg/mL aprotinin). For each cell sample, 30˜50 μg of total protein, along with a molecular weight ladder were resolved on 8% a bis-tris polyacrylamide NuPAGE gels (90˜130 V, 2 hrs) via electrophoresis and then transferred onto a nitrocellulose membrane (Invitrogen) (550 mA, 90 min) The membrane was first blocked using phosphate buffered saline (PBS) with 5% dry skimmed milk powder for 1 hr at room temperature and then incubated at 4° C. in the presence of mouse anti-serotonin transporter antibody (MAB1564; Chemicon), rabbit anti-norepinephrine transporter antibody (AB2234; Millipore, Bedford, Mass.), or anti-β-actin antibody (MAB1501, Chemicon) overnight. The antibody was diluted in phosphate buffered saline containing 0.1% Tween-20 (PBST). Afterwards, the membrane was washed with PBST and then incubated with a peroxidase-conjugated secondary antibody in PBST. After being washed for several times, the membrane was subjected to the enhanced chemiluminescence (ECL) analysis, following the method described in Yeh et al., 2009, Glia, 57:454-464, using an ECL kit purchased from Santa Cruz Biotechnology and Kodak X-OMAT LS film (Eastman Kodak, Rochester, N.Y.).

The results obtained from this study indicate that NRG1β decreased the levels of serotonin transporter in A172 cells, SH-SY5Y cells, primary astrocytes, and primary neurons in a dose-dependent manner (treatment with 10 ng/mL NRG1β: 0.45±0.08-fold, 0.34±0.05-fold and 0.55±0.07-fold of control for A172, primary astrocyte and primary neuron, respectively. p<0.05). NRG1β was also found to reduce the levels of norepinephrine transporter in both SH-SY5Y cells and primary neurons in a dose-dependent manner. As compared to control cells, the level of norepinephrine transporter was reduced to 0.58±0.07-fold lower in cells treated with 10 ng/mL NRG1β.

### (ii) Serotonin and Serotonin Receptor Agonists Increased Expression of ErbB4 and NRG1 in A172 Cells

A172 cells were cultured in the presence of serotonin at a concentration of 1, 3, 10, 30, or 100 μM. The levels of both NGR1 and ErbB4, the major receptor of NRG1 in the brain, in the treated A172 cells were examined by Western blot as described above at various time points (e.g., 2 hr, 4 hr, 8 hr, 12 hr, and 24 hr post treatment), using rabbit anti-NRG1 precursor antibody no. 07494 from Upstate® and rabbit anti-ErbB4 antibody sc-283 obtained from Santa Cruz Biotechnology (CA).

The results obtained from this study indicate that serotonin increased expression of both NRG1 and ErbB4 in a dose-dependent manner. At the serotonin concentration of 10 μM, the levels of NRG1 and ErbB4 were 1.4±0.06-fold and 1.7±0.1-fold, respectively, as compared to a saline control (p<0.05). At this concentration, the highest ErbB4 expression was observed 4 hours post treatment.

Next, A172 cells were treated with 5-HT_{1A} receptor agonist 8-hydroxy-N,N-dipropyl-2-aminotetralin (8-OH DPAT) at various concentrations (i.e., 0.1 μM, 0.3 μM, 1 μM, and 3 μM) or 5-HT_{2A} receptor agonist 2,5-dimethoxy-4-iodophenyl)-2-aminopropane (DOI) at various concentrations (i.e., 0.1 μM, 0.3 μM, 1 μM and 3 μM) for 4 hours. The levels of ErbB4 and NRG1 were examined by Western blotting, using rabbit anti-ErbB4 antibody sc-283 (purchased from Santa Cruz Biotechnology, Calif.) and rabbit anti-NRG1 precursor antibody from Upstate® (no. 07494). The results thus obtained indicate that 8-OH DPAT increased the levels of both ErbB4 and NRG1 in a dose-dependent manner (P<0.05). The maximal effect was observed at 0.3 μM 8-OH DPAT (1.44±0.10-fold and 1.44±0.12-fold for the levels of ErbB4 and NRG1, respectively, as compared with those in untreated A172 cells.) By contrast, A172 cells treated with DOI did not show increased levels of both ErbB4 and NRG1. These data indicate that serotonin-induced up-regulation of ErbB4 and NRG1 was mediated by the 5-HT_{1A} receptor, not the 5-HT_{2A} receptor.

### (iii) NRG^{+/-} Mice Exhibited Elevated Levels of Serotonin Transporter and Norepinephrine Transporter

Brain tissue samples obtained from different brain compartments of NRG1+/− mice were homogenized. The cell lysates thus obtained were collected and subjected to Western blot analysis to examine the levels of serotonin transporter (SERT) and norepinephrine transporter (NET), using mouse anti-SERT antibody MAB1564 from Chemicon and rabbit anti-NET antibody AB2234 from Millipore, Bedford, Mass.

As compared with their wild counterparts, the NRG1+/− mice exhibited an elevated level of SERT in frontal cortex, cortex, amygdala, dorsal hippocampus, ventral hippocampus and striatum and an elevated level of NET only in amygdala.

Taken together, the above-described results demonstrate that NRG1 modulates responsiveness to serotonin/norepinephrine transporter inhibitors. More specifically, a subject carrying a defective NRG1 gene is more sensitive to such inhibitors.

### Example 2

### Effects of Serotonin Transporter Inhibitors on NRG1^{+/-} and Wild-Type Mice

Transmembrane (TM)-domain NRG1 heterozygous mutant mice (129S5-Nrg1tm1Lex, ID#011745-UCD) were obtained from Mutant Mouse Regional Resource Centers (MMRRC) and backcrossed with B6 mice. NRG1^{+/-} mice and their wild-type littermates were generated by in-house mating of male NRG1^{+/-} mice and female C57BL/6 mice. Heterozygous crossing has been practiced for at least 7 generations to obtain animals with a homogeneous C57BL/6 genetic background except for the TM-mutated nrg1 gene locus. Mouse genotypes were determined by PCR analysis. Animals were housed (five per cage) under a 12-hr light/dark cycle (lights on from 8:00 a.m. to 8:00 p.m.) at constant room temperature and relative humidity with food and water available ad libitum. All experiments were started at 10-week-old period at weight of 25-30 g.

Both wild-type and NRG1^{+/-} mice were injected intraperitoneally with freshly prepared desipramine hydrochloride (Sigma, St. Louis, Mo.) at 20 mg/kg, twice a day for four days, following the method described in Kozisek et al., Neuropharmacology 54:251-257; 2008). Control mice were injected with a saline vehicle. The treated mice were sacrificed 2-4 hrs after the last injection, their brains removed various compartments separated, including frontal cortex, cortex, amygdala, dorsal hippocampus, ventral hippocampus, and striatum. The brain tissue samples were homogenized and centrifuged. The supernatants were collected and analyzed by Western blotting to examine levels of ErbB4, following the method described in Example 1 above. The results thus obtained show that the ErbB4 expression was increased for 1.47±0.2-fold in frontal cortex of desipramine-treated mice as compared with that in the control mice (P<0.05) and increased for 1.3±0.1-fold in amygdala of the treated mice as compared with that in the control mice (P<0.01).

Two behavior studies, i.e., the forced swimming test and social withdrawal behavior evaluation, were performed to study the effect of serotonin transporter inhibitors desipramine (20 mg/kg), imipramine (15 mg/kg; Sigma, St. Louis, Mo.), venlafaxine (15 mg/kg; Sigma, St. Louis, Mo.), duloxetine (20 mg/kg; Cymbalta®, Eli Lilly); fluvoxamine (15 mg/kg; Sigma, St. Louis, Mo.) and esticalopram (30 mg/kg; Lexapro®, Lundbeck) in NRG1^{+/-} mice or wild-type mice as follows.

Before undergoing any behavioral test, animals were handled by an investigator for at least 3 days so that they became familiar with the investigator. The animals had been placed under laboratory conditions for at least 1 hr before each behavioral test.

To perform the forced swimming test, each mouse, administered via intraperitoneal injection with either one of the above-listed serotonin transporter inhibitors or saline 30-60 minutes ago, was placed individually in a clear glass cylinder (height, 25 cm; diameter, 15 cm) containing water (depth: 15 cm; temperature: 26±1° C.). Its behavior was recorded by a video camera during a 6-min testing period. The duration of immobility was measured during the last 4 min after a 2-min habituation period.

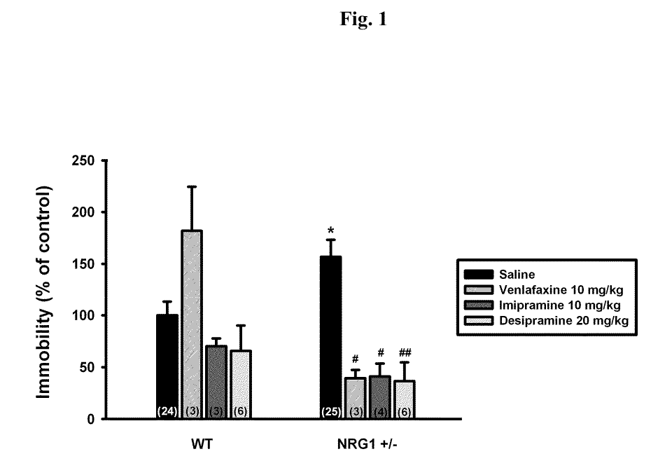

As shown in FIG. 1, NRG1^{+/-} mice displayed a longer immobility time than wild-type mice. Venlafaxine, imipramine and desipramine only slightly affected the immobility time of wild-type mice. Differently, all of the three SRIs decreased the immobility time of NRG1^{+/-} mice at degrees much greater than their wild-type counterparts. More specifically, the immobility periods of the control wild-type mice and the control NRG1+/− mice were 100.0±14.1% (n=20) and 122.8±15.0% (n=21), respectively; those of desipramine-treated wild-type mice and desipramine-treated NRG1+/− mice were 65.7±24.5% 4 (n=6; p=0.24) and 36.5±18.0% (n=6; p<0.01), respectively. These results indicate that NRG1+/− mice are more sensitive to desipramine in improving immobility. These types of mice were also found to be more sensitive to other SRIs of imipramine and venlafaxine in behavior changes.

A higher dosage of desipramine (50 mg/kg) was used to decrease immobility in C57BL/6 wild-type mice (101.4±16.4 sec vs. 58.6±11.0 sec for saline and desipramine, respectively).

The social withdrawal behavior evaluation study was performed following the method described in Sankoorikal et al., Biol. Psychiatry 59:415-423, 2006 with modifications. Briefly, this study was carried out in a behavioral testing apparatus containing two end chambers and one middle chamber. A clear Plexiglas cylinder was placed in each of the two end chambers, one being designated as “social side” (where stimulus to mice was introduced through the cylinder) and the other being designated as the “nonsocial side.” (where the cylinder is empty). Multiple holes (0.5 in diameter each) are evenly spaced over the surface of the two cylinders.

Each of the mice to be tested was housed individually for 3 days before the social withdrawal behavior evaluation study was performed. During the study, the response (i.e., social approach) of a test mouse to a novel (unfamiliar) “stimulus” was observed within 5 minutes after the mouse was exposed to the stimulus. A “social approach score” was calculated for each mouse according to the time it spent in each of the three chambers as follows: +1 for each second spent in the social side chamber, 0 for each second spent in the center chamber, and −1 for each second spent in the nonsocial side chamber. The “social approach change score” was calculated by subtracting the value of “social approach score in the absence of stimulus mouse” condition from the value for “presence of stimulus mouse” condition.

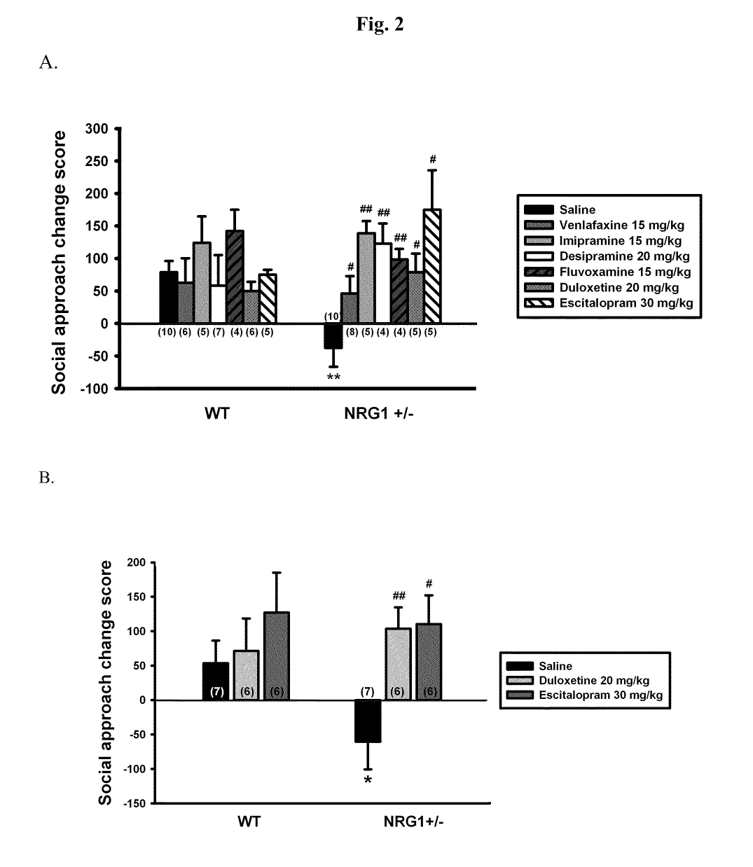

As shown in FIG. 2, panel A (male mice) and panel B (female mice), wild-type and NRG1^{+/-} mice showed significant difference in responding to an unfamiliar stimulus. More specifically, the social approach change score for the wild-type mice (i.e., male: 78.5±17.4, n=8; female: 53.6.5±32.8, n=7) was much higher than that for the NRG1^{+/-} mice (i.e., male: −37.7±28.8, n=10; female: −60.4±40.2, n=7), indicating that the wild-type mice take a much more robust approach toward a social stimulus than the NRG1^{+/-} mice.

SRIs of desipramine (20 mg/kg), imipramine (15 mg/kg), fluvoxamine (15 mg/kg), venlafaxine (15 mg/kg), duloxetine (20 mg/kg) and escitalopram (30 mg/kg) were injected (i.p.) 30 min before social testing. The social approach change score was not significantly different in wild-type mice. However, the social approach change scores were significantly increased by SRIs in NRG1+/− mice. The change scores are as following: desipramine 122.7±31.0 (n=4); imipramine 138.7±18.8 (n=5); fluvoxamine 98.6±15.9 (n=4); venlafaxine 61.9±24.7 (n=8); duloxetine [103.7±30.8 (n=6) and 78.7±28.5 (n=6) for female and male, respectively] and escitalopram [110.1±41.9 (n=6) and 175.3±60.4 (n=5) for female and male, respectively] (FIGS. 2A & 2B). (*p<0.05 compared with wild-type saline group. #p<0.05 compared with NRG1+/− mice saline group; ** or ##, p<0.01).

Taken together, the results obtained from this study indicate that NRG1+/− mice are more sensitive to SRI treatment as compared to their wild-type counterparts in behavior improvement. Thus, SRIs, selective norepinephrine reuptake inhibitor or 5-HT_{1A} agonists are suitable drugs for alleviating negative symptoms in schizophrenia patients who are defective in NRG1.

### Example 3

### Identifying a Defective NRG1 Gene by Examining a SNP

DNA was extracted from a blood sample of a candidate patient according to standard techniques. A fragment of the NRG1 gene, having the sequence of SEQ ID NO:1, was amplified by PCR using the following primers:

| Forward primer: | |
|---|---|
| GGCAGCTTTTCCTGCTTACA; | (SEQ ID NO: 2) |
| Reverse primer: | |
| TCTTTCCTGCAAAAGCTCAA. | (SEQ ID NO: 3) |

The PCR reaction was carried out in a 30 μl reaction mixture containing 75 ng genomic DNA, 7.5 pmol of each primer, 0.2 mM dNTPs, 3 U TEMPase Hot Start DNA Polymerase (Ampliqon) and 1×PCR buffer under the following conditions: an initial denaturation step at 94° C. for 15 min, followed by 30 cycles of denaturation at 94° C. for 45 sec, annealing 47.5° C. for 45 sec, extension at 72° C. for 30 sec with a final extension step at 72° C. for 7 min. The PCR products were purified by Gel/PCR DNA Fragments Extraction Kit (Geneaid) and subjected to sequencing analysis, using the BigDye® v3.1 Terminator sequencing kit (Applied Biosystems) and the ABI3730 automatic DNA sequencer (Applied Biosystems). The DNA sequence thus obtained was analyzed to determine the nucleotide at position 168 in SEQ ID NO:1. A T/C or C/C genotype at this SNP position indicates that the patient does not carry a risk NRG1 gene, while a T/T genotype indicates that the patient is NRG1 defective.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for alleviating a negative symptom in a schizophrenia patient comprising:
identifying a schizophrenia patient carrying a defective neuregulin 1 gene, and
administering to said patient a pharmaceutically effective amount of a compound that is a serotonin transporter inhibitor, a selective norepinephrine reuptake inhibitor, or a 5-HT_{1A} receptor agonist.

2. The method of claim 1, wherein the compound is a serotonin transporter inhibitor selected from the group consisting of citalopram, dapoxetine, escitalopram, fluoxetine, fluvoxamine, indalpine, paroxetine, sertraline, zimelidine, desvenlafaxine, duloxetine, levomilnacipran, milnacipran, venlafaxine, amitriptyline, butriptyline, clomipramine, desipramine, dosulepin, doxepin, imipramine, lofepramine, nisoxetine, nomifensine, nortriptyline, protriptyline, sibutramine and trimipramine.

3. The method of claim 1, wherein the compound is a selective norepinephrine reuptake inhibitor selected from the group consisting of amineptine, atomoxetine, bupropion, dexmethylphenidate, mazindol, methylphenidate, reboxetine, nisoxetine and viloxazine.

4. The method of claim 1, wherein the compound is a 5-HT_{1A} receptor agonist selected from the group consisting of buspirone, flesinoxan, gepirone, and ipsapirone.

5. The method of claim 1, wherein the pharmaceutically effective amount is 10 to 600 mg/day.

6. The method of claim 1, wherein the compound is administered to the patient orally, parenterally, topically, rectally, nasally, buccally, or vaginally.

7. The method of claim 1, wherein the compound is administered to the patient by an implanted reservoir or by inhalation.

8. The method of claim 2, wherein the pharmaceutically effective amount is 10 to 600 mg/day.

9. The method of claim 2, wherein the compound is administered to the patient orally, parenterally, topically, rectally, nasally, buccally, or vaginally.

10. The method of claim 2, wherein the compound is administered to the patient by an implanted reservoir or by inhalation.

11. The method of claim 3, wherein the pharmaceutically effective amount is 10 to 600 mg/day.

12. The method of claim 3, wherein the compound is administered to the patient orally, parenterally, topically, rectally, nasally, buccally, or vaginally.

13. The method of claim 3, wherein the compound is administered to the patient by an implanted reservoir or by inhalation.

14. The method of claim 4, wherein the pharmaceutically effective amount is 10 to 600 mg/day.

15. The method of claim 4, wherein the compound is administered to the patient orally, parenterally, topically, rectally, nasally, buccally, or vaginally.

16. The method of claim 4, wherein the compound is administered to the patient by an implanted reservoir or by inhalation.

17. The method of claim 1, wherein the identifying step is performed by examining the sequence of the neuregulin 1 gene.

18. The method of claim 1, wherein the identifying step is performed by examining the promoter sequence of the neuregulin 1 gene.

19. The method of claim 1, wherein the identifying step is performed by examining the single nucleotide polymorphism at position 168 in SEQ ID NO:1.

20. The method of claim 1, wherein the identifying step is performed by examining the mRNA or protein level of neuregulin 1.

21. A method of identifying a schizophrenia patient whose negative symptoms can be treated by a compound that is a serotonin transporter inhibitor, a selective norepinephrine reuptake inhibitor, or a 5-HT_{1A} receptor agonist, said method comprising
examining the function of a neuregulin 1 gene in a schizophrenia patient displaying a negative symptom, and
assessing whether the negative symptom of the patient can be treated with the compound based on the neuregulin 1 gene function in the patient, wherein presence of a defective neuregulin 1 gene indicates that the negative symptoms of the patient can be treated the compound.
